(19) United States
(12) Patent Application Publication
(10) Pub. No.: US 2023/0277899 A1
(43) Pub. Date: Sep. 7, 2023

(54) EXERCISE ASSISTANCE SYSTEM, EXERCISE ASSISTANCE METHOD, AND EXERCISE ASSISTANCE PROGRAM

(71) Applicant: Nippon Telegraph and Telephone Corporation, Tokyo (JP)

(72) Inventors: Yujiro Tanaka, Tokyo (JP); Daichi Matsunaga, Tokyo (JP); Takuro Tajima, Tokyo (JP); Michiko Seyama, Tokyo (JP)

(21) Appl. No.: 18/006,826

(22) PCT Filed: Aug. 31, 2020
(86) PCT No.: PCT/JP2020/032844

§ 371 Date: Jan. 25, 2023

(51) Int. Cl. A63B 24/00

(52) U.S. Cl. CPC A63B 24/0075; A63B 2071/0625; A63B 2225/64; A63B 2225/66; A63B 2230/062; A63B 2230/208; A63B 2230/505

(57) ABSTRACT

An exercise assistance system includes an arithmetic circuit, a core temperature sensor that measures a core temperature of a subject, a heart rate sensor that measures a heart rate of the subject, and a display device. The arithmetic circuit is configured to cause the display device to present actions to be taken by the subject, based on the core temperature of the subject measured by the core temperature sensor and the heart rate of the subject measured by the heart rate sensor.

### CROSS-REFERENCE TO RELATED APPLICATIONS

This patent application is a national phase filing under section 371 of PCT application no. PCT/JP2020/032844, filed on Aug. 31, 2020, which application is incorporated herein by reference in its entirety.

### TECHNICAL FIELD

The present invention relates to an exercise assistance system, an exercise assistance method and an exercise assistance program, and more particularly, to an exercise assistance system, an exercise assistance method and an exercise assistance program, each of which is capable of providing information on how and when appropriate training should be taken.

### BACKGROUND

For effectively taking exercise or training, it is desirable to appropriately manage a frequency, intensity, duration and type of exercise and training. A management system for determining whether or not the exercise is appropriate and for providing information on an appropriate training session and timing adopts an exercise intensity as a reference index.

The exercise intensity corresponds to exercise load and/or fatigue, and is generally represented by a numerical value based on physical ability of an exercising subject. The exercise intensity may be represented by, for example, the amount of physical activities, including “METs” which indicate a rate at which a person expands energy with 1 as a baseline, representing the amount of energy used when a person is at rest, “rating of perceived exertion (RPE)” which is a quantitative level of exertion that a subject feels during physical activity, and the heart rate.

The intensity of aerobic exercise is calculated by oxygen intake or heart rate with the maximum oxygen intake or maximum heart rate of the subject as a baseline. While measuring oxygen intake requires a complicated device, it is common to determine the exercise intensity using the heart rate that can be easily measured with a heart rate monitor since the heart rate increases linearly in proportion to the oxygen intake during exercise (see PTL 1 and PTL 2).

### Citation List

### Patent Literature

[PTL 1] Japanese Patent Application Laid-open No. 2019-154482.

[PTL 2] WO 2019/146616.

### Non Patent Literature

[NPL 1] Ming, et al. “Improvement of the dual-heat-flux method for deep body temperature measurement based on a finite element model.” 35th Annual International Conference of the IEEE Engineering in Medicine and Biology Society (EMBC). IEEE, 2013.

### SUMMARY

### Technical Problem

However, the heart rate varies significantly depending on the individual, thus it is not easy to accurately calculate the exercise intensity using the heart rate. Therefore, it is difficult to provide information on an appropriate training session or timing based on the exercise intensity when the subject takes exercise or training.

Embodiments of the present invention provide an exercise assistance system, an exercise assistance method and an exercise assistance program, each of which is capable of providing information on an appropriate training session and timing for a subject who takes exercise or training while suppressing fluctuations in exercise intensity due to individual differences.

### Solution to Problem

For achieving the object of embodiments of the present invention, the exercise assistance system according to embodiments of the present invention includes an arithmetic circuit; a first sensor that measures a core temperature of a subject; a second sensor that measures a heart rate of the subject; and an information presentation device. The arithmetic circuit is configured to cause the information presentation device to present actions to be taken by the subject, based on the core temperature of the subject measured by the first sensor and the heart rate of the subject measured by the second sensor.

In one embodiment of the exercise assistance system according to embodiments of the present invention, the exercise assistance system further includes a warming/cooling device that warms or cools the subject’s body. The arithmetic circuit is further configured to serve as a control circuit for controlling the warming/cooling device, based on the core temperature of the subject measured by the first sensor and the heart rate of the subject measured by the second sensor.

In one embodiment of the exercise assistance system according to embodiments of the present invention, the arithmetic circuit may be configured to cause the information presentation device to present warming-up and cooling-down instructions based on the core temperature of the subject measured by the first sensor.

The exercise assistance method according to embodiments of the present invention includes a first step of measuring a core temperature of a subject; a second step of measuring a heart rate of the subject; and a third step of causing an information presentation device to present actions to be taken by the subject, based on the core temperature and the heart rate of the subject.

In one embodiment of the exercise assistance system according to embodiments of the present invention, the third step includes a fourth step of controlling a warming/cooling device worn by the subject based on the core temperature and the heart rate of the subject, thereby warming or cooling the subject’s body.

In one embodiment of the exercise assistance system according to embodiments of the present invention, the third step may include a fifth step of causing an information presentation device to present warming-up and cooling-down instructions based on the core temperature of the subject.

The exercise assistance program according to embodiments of the present invention is a program causing a computer including an arithmetic circuit and a recorder to execute the exercise assistance method described above.

### Advantageous Effects of Embodiments of the Invention

According to the present invention, it is possible to provide information on an appropriate training session and timing for a subject who takes exercise or training while suppressing fluctuations in exercise intensity due to individual differences.

### BRIEF DESCRIPTION OF THE DRAWINGS

FIG. 1 is a diagram illustrating an exercise assistance system according to an embodiment of the present invention.

FIG. 2 is a diagram illustrating a configuration of an exercise assistance system according to an embodiment of the present invention.

FIG. 3A is a functional block diagram of an exercise assistance system according to an embodiment of the present invention.

FIG. 3B is a functional block diagram illustrating a modified example of an exercise assistance system according to an embodiment of the present invention.

FIG. 4A is a flowchart illustrating operations of an exercise assistance system according to an embodiment of the present invention.

FIG. 4B is a flowchart illustrating operations of an exercise assistance system according to an embodiment of the present invention.

FIG. 5 is a diagram illustrating a relationship between a core temperature and operations of an exercise assistance system according to the present embodiment.

### DETAILED DESCRIPTION OF ILLUSTRATIVE EMBODIMENTS

Hereinafter, embodiments of the present invention will be described in detail with reference to the drawings.

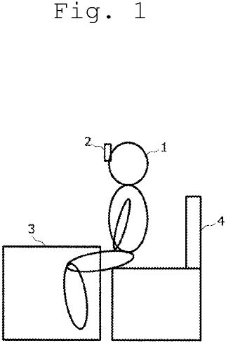

FIG. 1 shows that a subject 1 sits on a chair 4 while using an exercise assistance system 200 according to the present embodiment.

The exercise assistance system 200 according to the present embodiment includes an exercise assistance device 2 which is connected to various sensors for detecting, for example, a core temperature and a heart rate of the subject 1 who takes training, and presents actions to be taken by the subject 1 based on the detected core temperature and the heart rate. The system also includes a warming/cooling device 3 for warming or cooling the body of the subject 1.

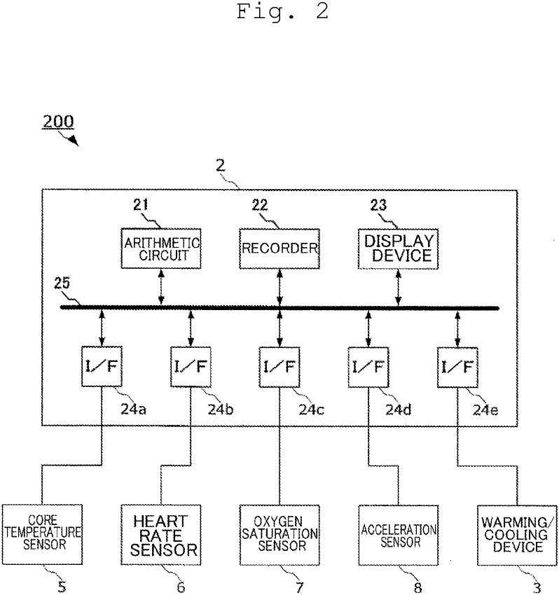

The exercise assistance device 2 can be implemented by a computer device in which an arithmetic circuit 21, a recorder 22, a display device 23, and interface (I/F) circuits 24a to 24e are communicatively connected to each other via a bus 25, or alternatively, a computer program (exercise assistance program) which is stored in a recorder 22 and causes the computer to operate as the exercise assistance device 2, as shown in FIG. 2. The recorder 22 may include a memory such a random access memory (RAM) or a read only memory (ROM), as well as an external storage device such as a solid state drive (SSD) or a hard disk drive (HDD). As the display device 23, an information presentation device including a liquid crystal panel and an organic electroluminescence (EL) display can be used.

A core temperature sensor 5 and a heart rate sensor 6 are connected to the exercise assistance device 2.

The core temperature sensor 5 is a sensor for measuring a temperature of a portion located below a certain depth from the epidermis of the subject 1 toward the core, also known as a deep body temperature or a core temperature. The core temperature can be determined by measuring a heat flux from detection results of a thermistor and a thermopile provided on a body surface of the subject 1, as described in, for example, NPL 1, or alternatively, by detecting infrared rays radiated from an eardrum or the vicinity thereof.

The heart rate sensor 6 is a device for counting heart beats per unit time. For example, the heart rate sensor 6 may be a chest pulse sensor for measuring electrical activity (ECG) of the heart via an electrode arranged on the chest and calculating a heart rate from the signal; or alternatively, an optical pulse sensor for measuring changes in a size of a blood vessel using photo plethysmography (PPG) with a light source such as an LED and a photodetector, and calculating a heart rate from the result.

The exercise assistance system 200 according to the present embodiment may be configured by connecting the exercise assistance device 2, in addition to the core temperature sensor 5 and the heart rate sensor 6, to an oxygen saturation sensor 7 for measuring oxygen saturation indicating how much oxygen is contained in blood in order to identify physiological conditions of the subject 1 and an acceleration sensor 8 for identifying motions of the subject 1, thereby identifying conditions of the subject 1 using values measured by those sensors.

On the other hand, a warming/cooling device 3 is a device for warming and cooling the body of the subject 1. The warming/cooling device 3 may be, for example, a bathtub provided with a refrigerant or a heater which is capable of warming or cooling from toes to thighs of the subject 1; or alternatively, an electric element such as a heater or a Peltier element which is incorporated in trousers or boots and can be attached on the legs of the subject. As will be described later, the exercise assistance device 2 also functions as a control device for the warming/cooling device 3.

In FIG. 1, the exercise assistance device 2 is attached to the head of the subject 1, for example, the forehead. However, as long as required data can be acquired by various sensors stated above, it is not necessarily required to attach the exercise assistance device 2 to the head of the subject 1.

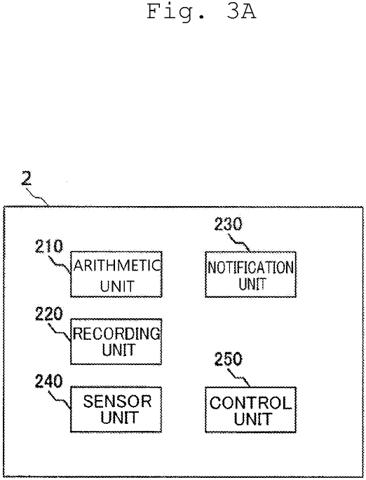

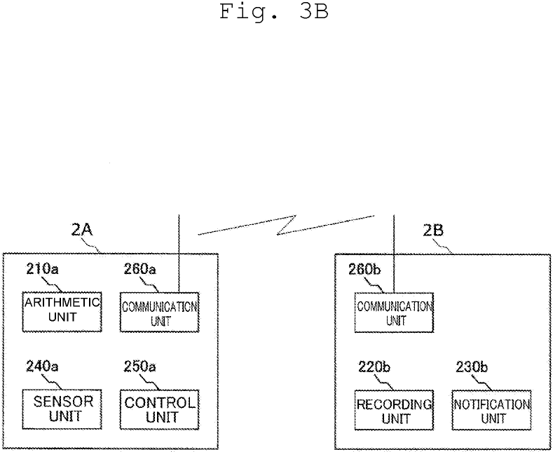

Functions of the exercise assistance device 2 will be described referring to a functional block diagram illustrated in FIG. 3A.

The exercise assistance device 2 includes an arithmetic unit 210, a recording unit 220, a notification unit 230, a sensor unit 240 and a control unit 250.

The arithmetic unit 210 is configured by an arithmetic circuit such as a central processing unit (CPU), and executes, according to the exercise assistance program, processing data acquired from the various sensors to calculate an exercise intensity or activity amount of the subject 1; determines whether the core temperature and the heart rate fall within a proper range; and notifies, via the notification unit 230, the subject 1 or their trainer of information on an appropriate training session or timing based on the core temperature and the exercise intensity.

The recording unit 220 includes the recorder 22 such as the internal memory and the external storage device, and records data measured by the various sensors, especially the core temperature and the heart rate. The recording unit 220 stores the exercise assistance program for causing the computer to serve as the exercise assistance device 2.

The notification unit 230 includes an information presentation device including, for example, the liquid crystal panel stated above, and displays information related to appropriate training session and timing. The notification unit 230 may be configured to include a voice device for outputting various information in voice.

The sensor unit 240 includes various sensors such as the core temperature sensor 5 and the heart rate sensor 6, and acquires data representing physiological and physical states of the subject 1 such as the core temperature and the heart rate.

The control unit 250 controls operations of the warming/cooling device 3.

In the examples shown in FIG. 2 and FIG. 3A, the exercise assistance device 2 has a structure in which each component is housed in one casing, but it is not necessarily required to be a single device. For example, as shown in FIG. 3B, a part of functions, for example, an arithmetic unit 210a, a sensor unit 240a and a control unit 250a may be incorporated into a main body 2A, and other functions, for example, a recording unit 220b and a notification unit 230b may be embedded in an external terminal device 2B. As the external terminal device 2B, for example, a smart device or a personal computer may be adopted. In this case, the main body 2A and the external terminal device 2B may be configured by respectively including a communication unit 260a and a communication unit 260b, such that data acquired by the sensor unit 240 of the main body 2A is transmitted from the main body 2A to the external terminal device 2B by a radio communication protocol such as Bluetooth®.

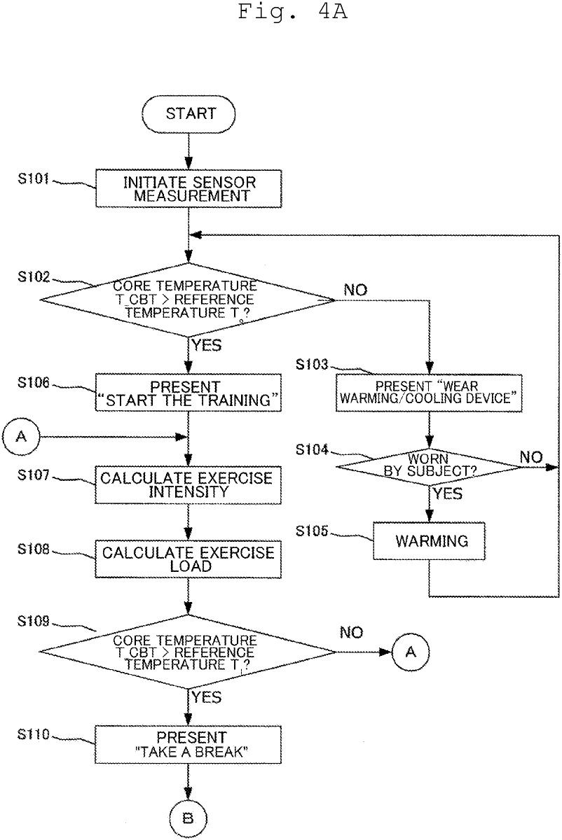

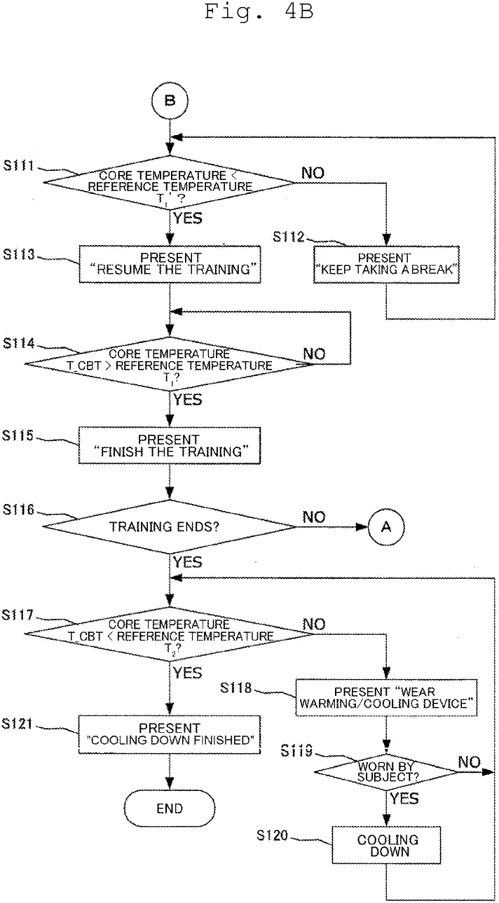

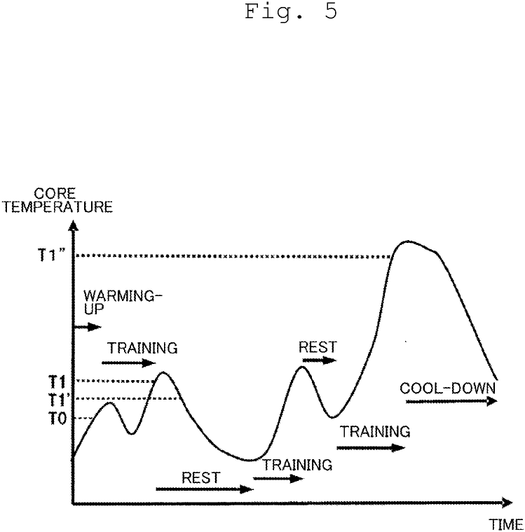

The operations of the exercise assistance system 200 according to the present embodiment will be described referring to FIGS. 4A, 4B and 5.

When the subject 1 who takes training wears the exercise assistance device 2 and starts the exercise assistance program, various sensors initiate measurement (S101). A first period is a period for warming up. The arithmetic unit 210 determines whether a core temperature T_CBT of the subject 1 measured by the core temperature sensor 5 exceeds a predetermined first reference temperature To, which is a baseline when the subject 1 should start the training, by comparing the core temperature T_CBT with the first reference temperature To (S102). Instead of the core temperature T_CBT measured by the core temperature sensor 5, an estimated core temperature T_est estimated by, for example, an artificial intelligence (AI) from the trend of the core temperature T_CBT may be compared with the first reference temperature To.

When the core temperature T_CBT does not exceed the first reference temperature To (S102: NO), the arithmetic unit 210 causes the notification unit 230 to present a message for urging the subject 1 to wear the warming/cooling device 3 (S103). When the subject 1 wears the warming/cooling device 3 (S104:YES), the warming/cooling device 3 is activated to warm the subject 1 until their core temperature exceeds the first reference temperature To (S105). In a case where it cannot be confirmed whether or not the subject 1 wears the warming/cooling device 3 (S104: NO), the exercise assistance device 2 waits for the core temperature T_CBT to exceed the first reference temperature To without activating the warming/cooling device 3.

When the core temperature T_CBT exceeds the first reference temperature To (S102: YES), it is considered that warming up has shown sufficient effect, thus the arithmetic unit 210 causes the notification unit 230 to present a message for urging the subject 1 to take off the warming/cooling device 3 and start the training (S106). During the training, the exercise assistance device 2 calculates the exercise intensity (S107) or the exercise load (S108) based on data acquired from various sensors, while determining whether or not the core temperature T_CBT exceeds a predetermined second reference temperature T1, which is a baseline when the subject 1 should rest (S109).

If the core temperature T_CBT does not exceed the second reference temperature T1 (S109: NO), the process returns to S107, and the training is continued until the core temperature T_CBT exceeds the second reference temperature T1. When the core temperature T_CBT exceeds the second reference temperature T1 (S109: YES), the arithmetic unit 210 causes the notification unit 230 to present a message for urging the subject 1 and/or their trainer to take a break (S110).

Thereafter, the exercise assistance device 2 determines whether or not the core temperature T_CBT falls below a predetermined third reference temperature T1′ (T1′ < T1) (S111). If the core temperature T_CBT does not fall below the third reference temperature T1′ (S111: NO), the arithmetic unit 210 causes the notification unit 230 to present a message for urging the subject 1 and/or their trainer to keep taking a break (S112) until the core temperature T_CBT falls below the third reference temperature T1′. When the core temperature T_CBT exceeds the third reference temperature T1′ (S111: YES), the arithmetic unit 210 causes the notification unit 230 to present a message for urging the subject 1 and/or their trainer to resume the training (S113).

After the subject 1 resumes the training, the exercise assistance device 2 determines whether or not the core temperature T_CBT exceeds a predetermined fourth reference temperature T1″ (> T1′) (S114). If the core temperature T_CBT does not exceed the fourth reference temperature T1″ (S114: NO), the exercise assistance device 2 waits until the core temperature T_CBT exceeds the fourth reference temperature T1″. When the core temperature T_CBT exceeds the fourth reference temperature T1″ (S114: YES), the arithmetic unit 210 causes the notification unit 230 to present a message for urging the subject 1 and/or their trainer to finish the training (S115).

In a case where the subject 1 does not finish the training at this time (S116: NO), the training is repeatedly stopped (taking a break) and resumed based on the second reference temperature T1, the third reference temperature T1′ and the fourth reference temperature T1″, from S107 to S113, while the subject 1 keeps taking the training. On the other hand, when the subject 1 finishes the training (S116: YES), for so-called cooling down, the exercise assistance device 2 determines whether the core temperature T_CBT falls below a predetermined fifth reference temperature T2 (< T1″) (S117). When the core temperature T_CBT does not fall below the fifth reference temperature T2 (S117: NO), the arithmetic unit 210 cause the notification unit 230 to display a message for urging the subject 1 to wear the warming/cooling device 3 (S118).

In a case where the subject 1 does not wear the warming/cooling device 3 at this time (S119: NO), the arithmetic unit 210 causes the notification unit 230 to keep presenting the message for urging the subject 1 to wear the warming/cooling device 3 until the core temperature T_CBT falls below the fifth reference temperature T2 (< T1″) (S118). When the subject 1 wears the warming/cooling device 3 (S119: YES), the control unit 250 activates the warming/cooling device 3 to cool the body of the subject 1 (S120). When the core temperature T_CBT falls below the fifth reference temperature T2 (< T1″), the arithmetic unit 210 causes the notification unit 230 to present a message for notifying the subject 1 or their trainer that cooling down is finished (S121), and the process ends.

As stated above, according to the exercise assistance system 200 of the present embodiment, it is possible to present the exercise intensity and duration appropriate for the subject 1 who takes training or the trainer supporting the training with high accuracy, and to improve the quality of the training.

By using the warming/cooling device 3, it is possible to create a state in which the core temperature falls within a predetermined temperature range in a limited time. For example, the core temperature can be suitably adjusted for warming up, cooling down and training.

By displaying the core temperature and the heart rate on the notification unit 230, information on them can be provided to, for example, the subject 1 and the trainer. Since the exercise intensity and the exercise load are calculated from the core temperature and the heart rate, and displayed on the notification unit 230, information on the exercise performance based on the core temperature can be provided to, for example, the subject 1 and their trainer. Thus, it is possible for the subject 1 and the trainer to know whether training is properly taken or not.

By displaying a future estimated core temperature T_est estimated by, for example, the AI from the trend of the core temperature T_CBT on the notification unit 230, information on the future core temperature can be provided to, for example, the subject 1 and their trainer.

Embodiments of the present invention have been described in detail above, but the present invention is not limited thereto. The specific configuration and details of embodiments of the present invention can be altered or modified in various manners which can be understood by those skilled in the art within the scope of the present invention.

The exercise assistance program according to the embodiments of the present invention may be provided by recording on a non-transitory computer-readable recording medium such as a recording device or CD-ROM, or may be provided via a network.

### INDUSTRIAL APPLICABILITY

Embodiments of the present invention can be used in the field of exercise assistance.

Reference Signs List

| | |
|---|---|
| 1 | Subject |
| 200 | Exercise assistance system |
| 2 | Exercise assistance device |
| 2A | Main body |
| 2B | External terminal device |
| 21 | Arithmetic circuit |
| 22 | Recorder |
| 23 | Display device |
| 24a to 24e | Interface |
| 210, 210a | Arithmetic unit |
| 220, 220b | Recording unit |
| 230, 230b | Notification unit |
| 240, 240a | Sensor unit |
| 250, 250a | Control unit |
| 260a, 260b | Communication unit |
| 3 | Warming/cooling device |
| 5 | Core temperature sensor |
| 6 | Heart rate sensor |
| 7 | Oxygen saturation sensory |
| 8 | Acceleration sensor |

1-7. (canceled)

8. An exercise assistance system comprising:
a first sensor;
a second sensor;
an information presentation device; and
an arithmetic circuit configured to cause the information presentation device to present actions to be taken by a subject based on a core temperature of the subject measured by the first sensor and a heart rate of the subject measured by the second sensor.

9. The exercise assistance system according to claim 8, further comprising a warming/cooling device configured to warm or cool the subject’s body.

10. The exercise assistance system according to claim 9, wherein the arithmetic circuit is configured to serve as a control circuit for controlling the warming/cooling device based on the core temperature of the subject measured by the first sensor and the heart rate of the subject measured by the second sensor.

11. The exercise assistance system according to claim 10, wherein the arithmetic circuit is configured to cause the information presentation device to present warming-up and cooling-down instructions based on the core temperature of the subject measured by the first sensor.

12. The exercise assistance system according to claim 8, wherein the arithmetic circuit is configured to cause the information presentation device to present warming-up and cooling-down instructions based on the core temperature of the subject measured by the first sensor.

13. The exercise assistance system according to claim 8, further comprising an oxygen saturation sensor configured to measure oxygen saturation indicating how much oxygen is contained in blood of the user.

14. The exercise assistance system according to claim 8, further comprising an acceleration sensor configured to identify motions of the subject.

15. An exercise assistance method comprising:
measuring a core temperature of a subject;
measuring a heart rate of the subject; and
causing an information presentation device to present actions to be taken by the subject based on the core temperature of the subject and the heart rate of the subject.

16. The exercise assistance method according to claim 15, further comprising controlling a warming/cooling device worn by the subject based on the core temperature and the heart rate of the subject to perform warming or cooling of the subject’s body.

17. The exercise assistance method according to claim 16, further comprising causing the information presentation device to present warming-up and cooling-down instructions based on the core temperature of the subject.

18. The exercise assistance method according to claim 15, further comprising causing the information presentation device to present warming-up and cooling-down instructions based on the core temperature of the subject.

19. A non-transitory computer readable medium for storing instructions that, when executed by a computer comprising an arithmetic circuit and a recorder, cause the computer to perform the exercise assistance method according to claim 13.

20. An exercise assistance device comprising:
a plurality of sensors;
a notification device; and
a computer comprising an arithmetic circuit and a recorder, wherein the computer is configured to:
execute processing data acquired from the plurality of sensors to calculate an exercise intensity or an activity amount of a subject;
determine whether a core temperature and a heart rate are within a predetermined range; and
provide information through the notification device on an appropriate training session or timing based on the core temperature or the exercise intensity.

21. The exercise assistance device according to claim 20, wherein the notification device comprises a display or a voice device.

22. The exercise assistance device according to claim 20, wherein the plurality of sensors comprises a core temperature sensor and a heart rate sensor.

23. The exercise assistance device according to claim 22, wherein the plurality of sensors further comprises an oxygen saturation sensor and an acceleration sensor.

24. The exercise assistance device according to claim 20, further comprising:
a warming/cooling device; and
a controller configured to control operations of the warming/cooling device.
